(19) United States
(12) Patent Application Publication
(10) Pub. No.: US 2019/0362853 A1
(43) Pub. Date: Nov. 28, 2019

(54) METHOD AND SYSTEM FOR ERGONOMIC AUGMENTATION OF WORKSPACES

(71) Applicant: APEX OCCUPATIONAL HEALTH SOLUTIONS INC., Kitchener (CA)

(72) Inventors: Kurt William FRIARS, Guelph (CA); Douglas FRIARS, Kitchener (CA); Jon HOWARD, Guelph (CA); Daniel WALDIE, Whitby (CA)

(21) Appl. No.: 16/484,974

(22) PCT Filed: Feb. 9, 2018
(86) PCT No.: PCT/IB2018/050814

§ 371 Date: Aug. 9, 2019

Related U.S. Application Data
(60) Provisional application No. 62/457,412, filed on Feb. 10, 2017.

(51) Int. Cl. G16H 50/30; G16H 10/20; A61B 5/00; G06N 20/10

(52) U.S. Cl. CPC G16H 50/30; G06N 20/10; A61B 5/4561; G16H 10/20

(57) ABSTRACT

A method and system for ergonomically augmenting a workspace. A contextual and interactive automated ergonomic assessment is performed by which user data relating to a workspace setup and a patient's experience of the workspace is obtained. Features and deficiencies of equipment in the workspace, and possible ergonomic risks to the patient, are identified. The system includes a relational library of equipment, equipment features, deficiencies, and ergonomic issues, which may be referenced to select recommended equipment to augment the workspace. Additional ergonomic interventions may also be recommended. A predictive model may gather insights and may be leveraged to make statistically-informed recommendations for augmentations to the workspace.

### CROSS-REFERENCE TO RELATED APPLICATIONS

This application claims priority to U.S. 62/457,412, filed Feb. 10, 2017, the entirety of which is incorporated herein by reference.

### FIELD

The present disclosure relates to ergonomics, and in particular to computerized tools for workspace ergonomics assessment, management, and education.

### BACKGROUND

Ergonomics is an important consideration in many workspace environments for improving individuals' wellbeing and productivity. Particularly with respect to office workspace environments, office workers are at risk of experiencing repetitive stress injuries and debilitating discomfort merely by frequently using everyday office equipment such as furniture computer systems.

In an effort to bolster healthy ergonomic workspaces, organizations have engaged in professional ergonomics assessment programs whereby trained ergonomics professionals educate and train workers on proper ergonomic use of office equipment, and select ergonomically suitable office equipment to suit a given workspace. Although a personal professional assessment may be effective, hiring such ergonomics professionals can be prohibitively expensive, especially at the scale of large organizations.

### SUMMARY

According to an aspect of the disclosure, a system for ergonomically augmenting a workspace includes a patient user interface for receiving workspace setup data and patient experience data, the workspace setup data including equipment data related to equipment used in a workspace, the patient experience data relating to patient use of said equipment, a memory, the memory storing programming instructions and a library of equipment data, the library of equipment data relating equipment identity and equipment features, and a processor in communication with the memory and configured to execute the programming instructions to execute a recommendation engine, wherein the recommendation engine is configured to associate the patient experience data with the workspace setup data in the library of equipment data to identify deficiencies of the equipment used in the workspace, and generate a recommended augmentation which ameliorates the deficiencies of the equipment used in the workspace.

According to another aspect of the disclosure, a method for augmenting a workspace involves maintaining a library of equipment data, the library of equipment data relating equipment identity and equipment features, receiving workspace setup data, the workspace setup data including equipment data related to equipment used in a workspace, receiving patient experience data, the patient experience data relating to patient use of said equipment, associating the patient experience data with the workspace setup data in the library of equipment data to identify deficiencies of the equipment used in the workspace, and generating a recommended augmentation which ameliorates the deficiencies of the equipment used in the workspace.

The memory may store a training library comprising workspace setup data and patient experience data, the training library relating workspace setup data to patient experience data and to outcomes of recommended augmentations, and the recommendation engine may generate a recommended augmentation according to a predictive model based at least in part on the training library.

The predictive model may include a machine learning model trained with the training library to recognize differential outcomes in patient experience data based at least on the outcomes of recommended augmentations. The machine learning model may include a support vector machine. The predictive model may include a machine learning model trained with the training library to predict ergonomic risk.

The patient experience data may include psychometric data, and the predictive model may be trained with the psychometric data to predict susceptibility to ergonomic risk. At least a portion of the psychometric data may be obtained by recording interaction with the patient user interface. The recommended augmentation may include psychological therapy for a patient using the workspace.

The recommended augmentation may include recommended equipment for the workspace having equipment features which ameliorate the deficiencies of the equipment used in the workspace.

The recommendation engine may be configured to exclude from recommendation the augmentation of recommended equipment which would introduce new deficiencies to the equipment used in the workspace.

The processor may be further configured to receive workspace setup data via a sensor device configured to identify equipment present in the workspace.

The recommended augmentation may include a recommended regimen of physical therapy for a patient using the workspace.

The memory may store a question library, and the patient user interface may include an interrogative component for outputting a question from the question library, the question directed to obtaining patient workspace setup data or patient experience data related to a piece of equipment used in the workspace from a patient, a graphical component for displaying a representation of the piece of equipment used in the workspace to which the question is related, and for displaying visual cues to guide the patient to answer the question, and an input component for receiving a response to the question.

The graphical component may display a representation of the workspace, the representation of the workspace generated from the workspace setup data, the graphical component further displaying a highlighted portion indicating the representation of the piece of equipment used in the workspace to which the question is related.

The interrogative component may be configured to output questions according to a branching sequence of questioning whereby a particular branch of questions is output depending on a previous answer to a previous question of a previous branch of questions.

The system may include a manager user interface, and the processor may be configured to display the workspace setup data and patient experience data and the recommended augmentation to the manager user interface.

The system may include a computer network, a network interface in communication with the processor, a patient device running the patient user interface and in communication with the network interface via the computer network, and a manager device running the manager user interface and in communication with the network interface via the computer network.

### BRIEF DESCRIPTION OF THE DRAWINGS

Non-limiting embodiments of the present disclosure will now be described, by way of example only, with reference to the attached Figures, wherein:

FIG. 1 is a schematic diagram of a system for ergonomically augmenting a workspace;

FIG. 2 is a block diagram of the functional components of a patient device of the system of FIG. 1;

FIG. 3 is a block diagram of the functional components of an assessment server of the system of FIG. 1;

FIG. 4A is a block diagram of the functional software modules of the patient device of FIG. 2;

FIG. 4B is a block diagram of the functional software modules of the assessment server of FIG. 3;

FIG. 5 is a schematic diagram of a recommendation engine executed by the assessment server of FIG. 3;

FIG. 6 is a flowchart showing a method for augmenting a workspace;

FIG. 7 is a schematic diagram showing a data structure layout of workspace setup data;

FIG. 8 is a schematic diagram showing a data structure layout of patient experience data;

FIG. 9 is a schematic diagram showing a data structure layout of feature equipment data;

FIG. 10 is a schematic diagram showing a data structure layout of equipment ergonomic data;

FIG. 11 is a schematic diagram showing a data structure layout of related deficiency data;

FIG. 12A is a schematic diagram of a patient user interface for gathering workspace setup data;

FIG. 12B is a schematic diagram of a patient user interface for gathering patient experience data;

FIG. 12C is a schematic diagram of a patient user interface for gathering psychometric data;

FIG. 12D is a schematic diagram of another patient user interface including visual cues to guide the patient;

FIG. 13 is a flowchart of a method for performing an ergonomic assessment of a patient using a workspace;

FIG. 14 is a branch diagram showing branching sequences of questions for performing an ergonomic assessment;

FIG. 15 is a flowchart showing a method of generating recommended equipment for a workspace; and

FIG. 16 is a schematic diagram of another embodiment of system for ergonomically augmenting a workspace, wherein the system includes a sensor device.

### DETAILED DESCRIPTION

With the advent of computers, and especially the internet, attempts have been made to create automated ergonomic assessment, management, and education tools, to meet the need to reduce assessment costs while still providing ergonomics services to organizations large and small. Such systems typically lack the ability to meaningfully educate users on proper ergonomics practice without assuming a certain degree of ergonomics expertise. Further, such systems typically focus on testing for ergonomics risks, without having the ability to assist a user directly given the equipment at hand. Moreover, such systems typically lack the ability to derive meaningful insights from previously performed ergonomic interventions and to make meaningful recommendations for how to address identified ergonomics risks with a wide variety of available ergonomic equipment and interventional techniques.

The present disclosure relates to ergonomics, and in particular to computerized tools for workspace ergonomics assessment, management, and education. In one aspect, this disclosure provides a system for ergonomically augmenting a workspace.

The system includes providing a workplace ergonomics assessment to a patient, which involves obtaining workspace setup data and patient experience data, including psychometric data, relating to the patient's use of the workspace from the patient through a user interface. By engaging in the ergonomics assessment, deficiencies in workspace equipment and possible ergonomics risks may be identified.

The assessment may involve providing a dynamically-selected sequences of interrogation contextually relevant to the particular patient and workspace so that irrelevant information is not captured. Further, the assessment may involve using a graphical component of a user interface to display representations of the patient's workspace, and workspace equipment therein, along with ergonomic education or instruction as to how to properly use the workspace equipment. The graphical component may include highlighted portions or visual cues to guide the patient through proper setup of the patient's ergonomic equipment or through a line of questioning.

The system includes a database hosting a library which links available equipment to equipment features, equipment deficiencies, possible related deficiencies, and other information relevant to managing and improving workplace ergonomics. Having gathered data relevant to an ergonomics assessment, the system can make recommendations to ameliorate such deficiencies and/or alleviate such risks. For example, the system may reference the database to determine an ergonomically appropriate set of equipment for the patient to use. More generally, the system may make recommended augmentations may include additional recommendations for additional or changed workspace equipment, such as furniture, and may include additional ergonomics interventions such as a recommended regimen of ergonomic therapy such as physical therapy.

The database can be leveraged to apply machine learning to generate recommendations for how to improve ergonomics in the workspace. For example, recommendations may include changes in equipment which may be predicted to improve ergonomics outcomes based on data relating to previous ergonomics assessments and intervention outcomes. Furthermore, the database can be leveraged to generate predictions of ergonomics risks, such that realization of workplace injuries can be preemptively avoided. Psychometric data may be used to predict a patient's susceptibility to ergonomic risk, including the risk that the patient will suffer an impairment such as a musculoskeletal injury, and the risk that such an impairment may manifest into a disability affecting the patient's use of the workspace.

Such recommendations and predictions can thus lead to cost savings for organizations using the system due to reduced worker injury, reduced absenteeism, and reduced insurance costs.

The system may be distributed across one or more devices and computer networks, which may include at least one patient device running software for executing the workspace ergonomics assessment, and an assessment server hosting the database and running software for executing the algorithms to make recommended equipment or interventional recommendations. The system may also include a manager device which allows widescale management of the ergonomics assessments and the recommendations made.

Detailed descriptions of specific embodiments are now presented with reference to the attached Figures. Although the following embodiments generally describe systems and methods for ergonomic augmentation of workspaces, it is to be understood that the example embodiments and explanations presented herein do not limit the scope of the present disclosure.

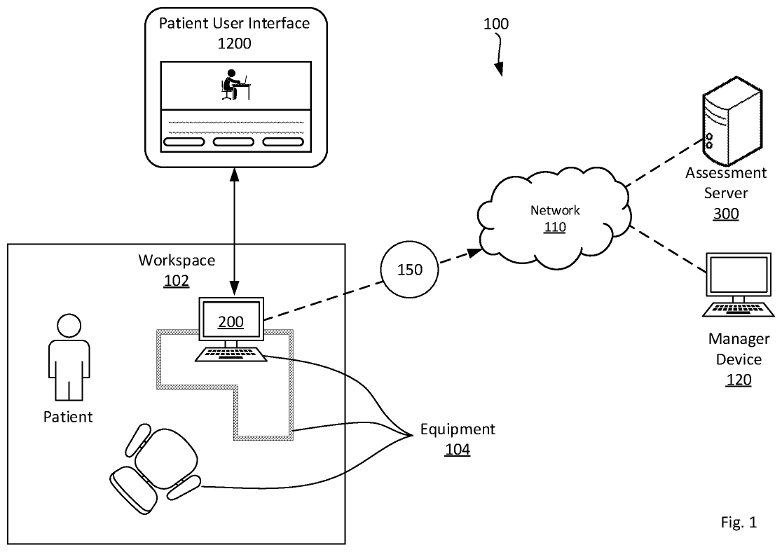

FIG. 1 is a schematic diagram of a system 100 for augmenting a workspace, according to a non-limiting embodiment. The system 100 includes a workspace, indicated generally at 102. The workspace 102 includes equipment 104 used by a patient, which in this example embodiment includes an office chair, and computer terminal. The computer terminal is indicated as patient device 200.

In addition to the patient device 200, the system 100 includes a manager device 120 and an assessment server 300, which each communicate over one or more computer networks, indicated as network 110. The manager device 120 runs a software application providing for a manager user interface for monitoring and management of ergonomics assessments and recommendations made by the assessment server 300.

The patient device 200 and manager device 120 each include a computing device running a user application with storage, communication, and processing means. Although termed a server in this example embodiment, the assessment server 300 includes a computing device running a user or server application with storage, communication, and processing means.

The network 110 can include the internet, a Wi-Fi network, a local-area network, a wide-area network (WAN), a wireless cellular data network, a virtual private network (VPN), a combination of such, and similar. Although a single assessment server 300 is described, it is understood that assessment server 300 may refer to a combination of computers and/or servers, such as in a cloud computing environment.

The patient device 200 runs a software application providing a patient user interface 1200 for delivering an ergonomics assessment to the patient. During the course of an ergonomics assessment, the patient device 200 transmits user data 150, which may include data relating to the setup of the workspace 102 and the equipment 104 therein, and which may further include data relating to the patient's experience using said workspace 102 and equipment 104, over network 110 to the assessment server 300 and manager device 120. For example, the user data 150 may include workplace setup data indicating that the patient's workspace is lacking a chair with proper lumbar support, and the patient experience data may indicate that the user experiences discomfort in the lower back 3-5 times per week. The assessment server 300 can use user data 150 to coordinate ergonomics assessments, make predictions to preemptively avert ergonomic risk, and make recommendations for augmentations to improve the ergonomic circumstances of the patient, as discussed throughout this disclosure.

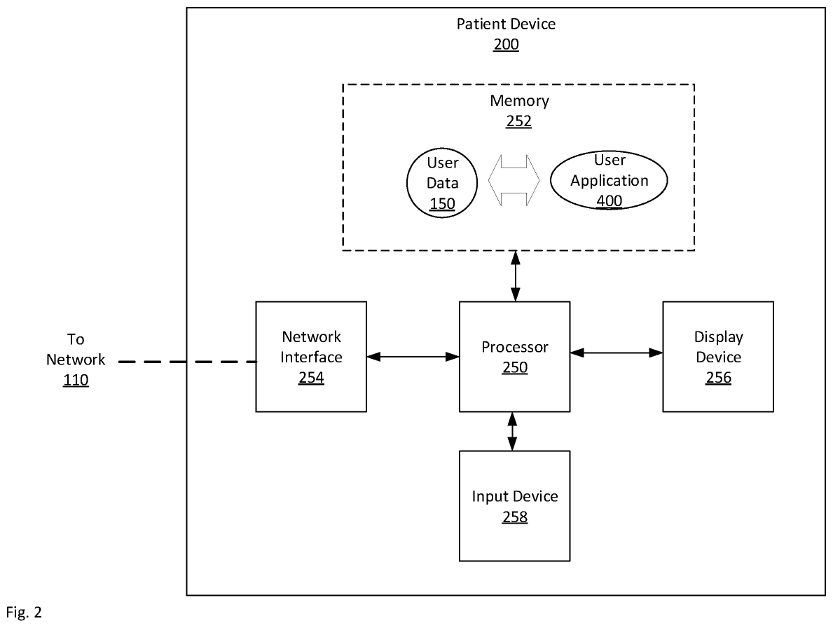

FIG. 2 is a block diagram of functional components of patient device 200, according to a non-limiting embodiment. Patient device 200 includes a processor 250, a memory 252, a network interface 254, a display device 256, and an input device 258.

Although a single processor 250 is shown, the term “processor” as discussed herein refers to any quantity and combination of a processor, a central processing unit (CPU), a microprocessor, a microcontroller, a field-programmable gate array (FPGA), and similar.

The network interface includes programming logic enabling the patient device 200 to communicate over network 110, is configured for bidirectional data communications through the network 110, and accordingly can include a network adaptor and driver suitable for the type of network used.

The memory 252 can include volatile storage and non-volatile storage. Volatile storage may include random-access memory (RAM) or similar. Non-volatile storage may include a hard drive, flash memory, and similar. The memory 252 stores the user data 150 for transmission to assessment server 300. The memory 252 further stores programming instructions for implementing user application 400, which provides the patient user interface 1200 and obtains the user data 150 from the patient.

The display device 256 includes a graphical display surface that can be configured to display patient user interface 1200.

The input device 258 converts actions of a user into commands and data inputted into the patient device 200. An input device 258 can include a keyboard, a mouse, or other pointing device. The input device 258 may be coupled with the display device 256 as a touch screen interface.

In some embodiments, the patient device 200 and/or the management device 120 may include a smart phone running an operating system such as, for example, Android®, iOS®, Windows® mobile, BB 10, or similar, or in other embodiments, a desktop computer, a tablet computer, a laptop, a desktop computer, or similar.

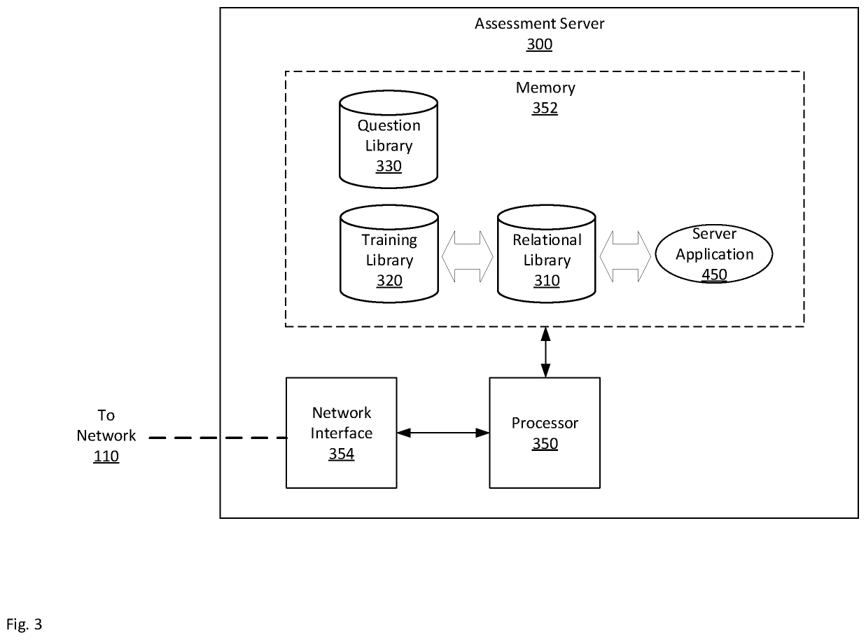

FIG. 3 is a block diagram of functional components of assessment server 300, according to a non-limiting embodiment. Assessment server 300 includes a processor 350, a memory 352, and a network interface 354. For description of the processor 350, memory 352, and network interface 354, reference may be had to analogous components of patient device 200.

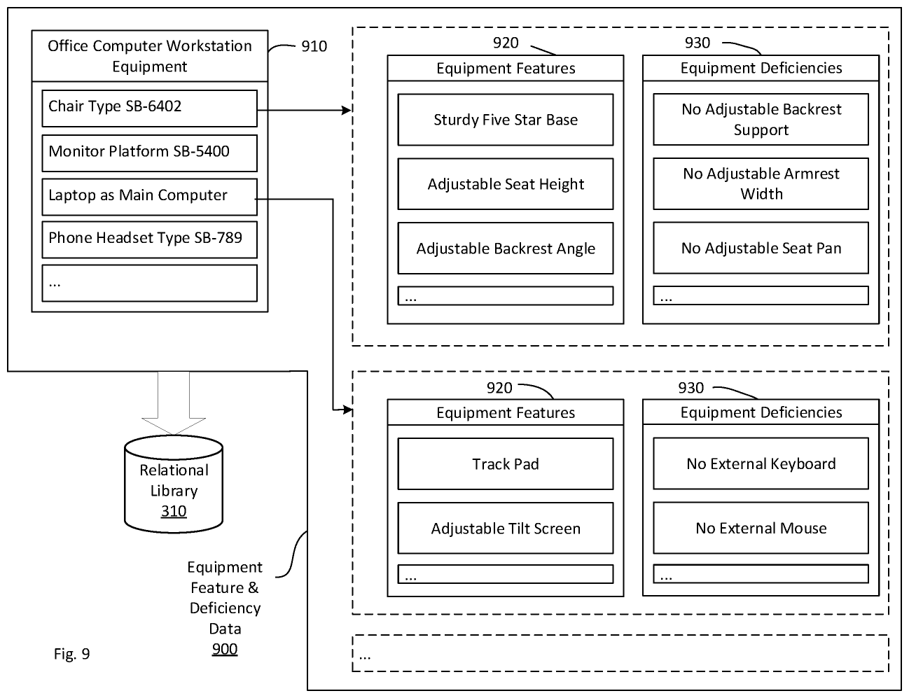

The memory 352 stores a relational library 310, a training library 320, and server application 450. The relational library 310 includes a library of equipment data which relates equipment identity to specific features and specific deficiencies of that equipment (see FIG. 9). The training library 320 includes previously compiled user data 150, and data relating to previous ergonomic assessments, which may be useful toward building a predictive model to aid in recommending augmentations to a workspace. The server application 450 includes software to coordinate ergonomics assessments, make predictions to preemptively avert ergonomic risk, and make recommendations for augmentations to improve the ergonomic circumstances of the patient, as discussed throughout this disclosure.

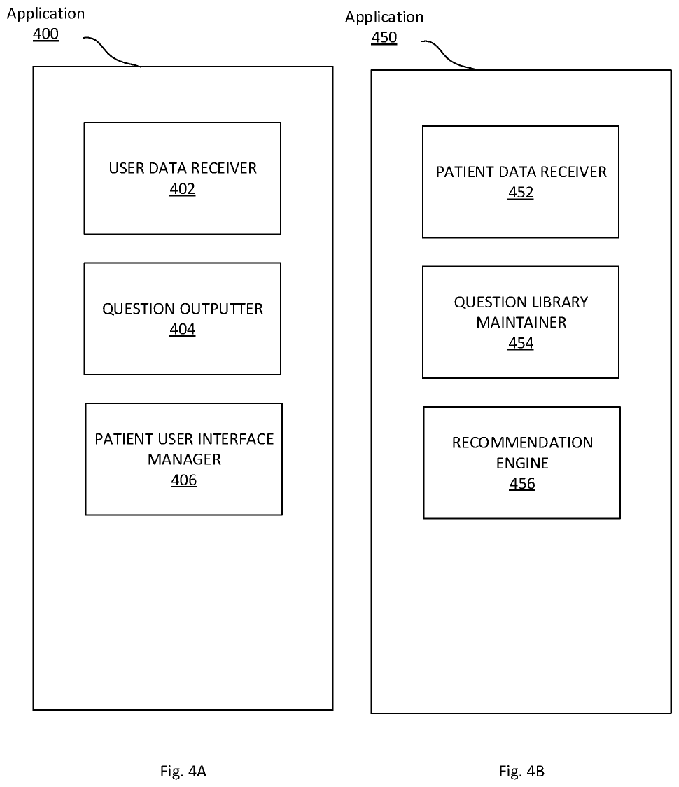

FIG. 4A is a block diagram of the functional modules of user application 400, according to a non-limiting embodiment. The user application 400 includes user data receiver 402, question outputter 404, and patient user interface manager 406.

User data receiver 402 is a program comprising programming instructions for receiving input through patient device 200, e.g. through input device 258, and relaying the input to patient user interface manager 406 for inclusion into user data 150.

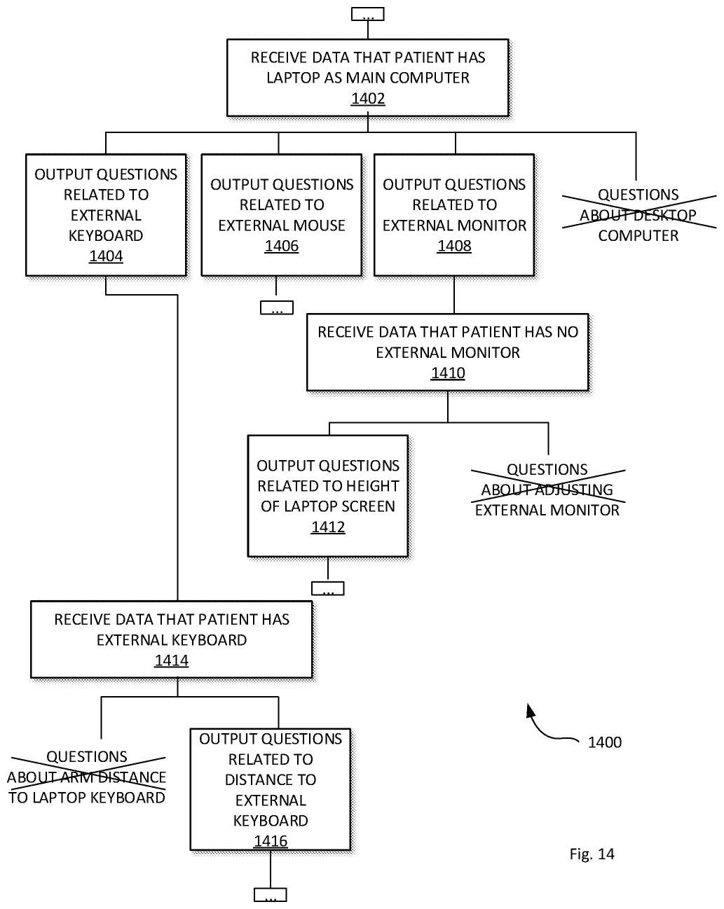

Question outputter 404 is a program comprising programming instructions for outputting questions via patient user interface 1200 to obtain user data 150 from the patient. The questions may provide a dynamically-selected sequences of questions contextually relevant to the particular patient and workspace, discussed in greater detail in FIG. 14.

Patient user interface manager 406 coordinates user data receiver 402, question outputter 404, and patient user interface 1200 to provide an ergonomics assessment to the patient. Patient user interface manager 406 may include logic to provide equipment adjustment recommendations directly to the user upon conclusion of an ergonomics assessment. For example, where the user has indicated a discomfort that is solvable with the equipment at hand, such as by adjusting a reclining angle of an office chair, the patient user interface manager 406 may directly output such a suggestion.

FIG. 4B is a block diagram of the functional modules of server application 450, according to a non-limiting embodiment. The server application 450 includes patient data receiver 452, question library maintainer 454, and recommendation engine 456.

Patient data receiver 452 is a program comprising programming instructions for cooperating with user application 400 to receive user data 150 from the patient user interface 1200 of the patient device 200 via network 110.

Question library maintainer 454 is a program comprising programming instructions for maintaining a question library 330, which is used for interrogation during the ergonomics assessment provided via patient user interface 1200. In some embodiments, the questions may be periodically transmitted to the patient device 200. In other embodiments, the questions may be pulled live from the assessment server 300 by the patient user interface manager 406.

Recommendation engine 456 is a program providing programming instructions to make recommendations to augment the patient's workspace. In some embodiments, the recommendation engine 456 associates patient experience data with workspace setup data included in the user data 150 to identify deficiencies or ergonomic risks associated with the equipment used in the patient's workspace, and thereby generates one or more recommended augmentations which ameliorate such deficiencies or reduce such risk. Methods for making such recommendations are described in greater detail below.

Although user application 400 and server application 450 are depicted as separate applications running on separate devices, it is to be understood that this is not limiting and that the duties of some of the functional modules therein can be arbitrarily distributed between the applications and/or devices.

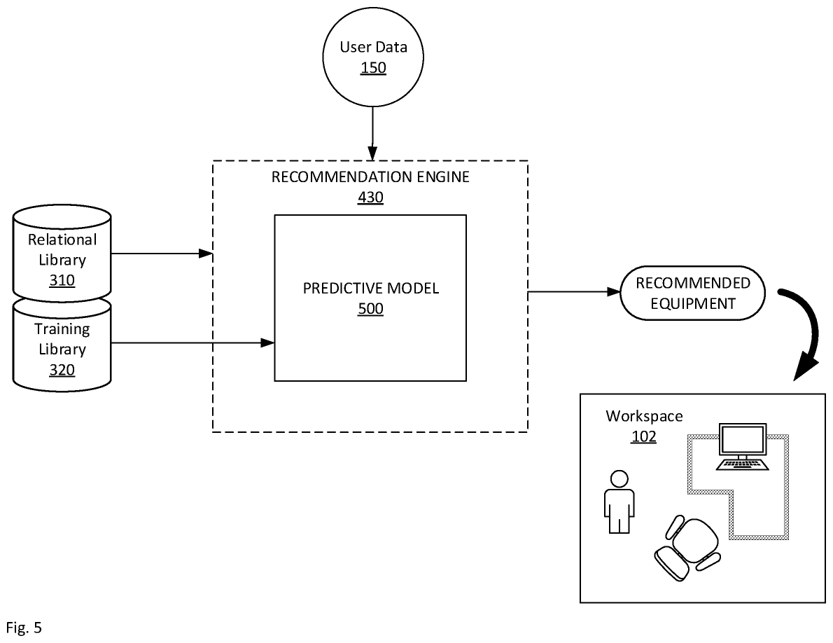

FIG. 5 is a schematic diagram of the recommendation engine 430, according to a non-limiting embodiment. Methods for recommending augmentations for a workspace are described in detail below, and can be read with reference to FIG. 5 in tandem for greater understanding. Briefly, the recommendation engine 430 receives user data 150, including information relating to the equipment setup of a workplace, including the equipment present and the equipment features and deficiencies, and information relating to the patient's experience in relation to using the equipment in the workspace. In some embodiments, the recommendation engine may reference the relational library 130 to algorithmically determine an ergonomically appropriate set of equipment for the patient to use.

In some embodiments, the determination of an ergonomically appropriate set of equipment is driven, aided, or complemented, by a predictive model 500 trained with training library 320. The training library 320 includes training data including data from previous ergonomics assessments and interventions. The training library 320 relates workspace setup data to patient experience data, and to outcomes of previous ergonomic interventions (e.g. previous recommendations of workspace equipment or previous regimens of physical therapy), which is incorporated into predictive model 500 to more accurately determine an ergonomically appropriate recommendation.

Thus, in some embodiments, the predictive model 500 is trained to recognize more beneficial and less beneficial outcomes to ergonomic augmentations, and to adjust the recommendation engine 430 to make recommendations of ergonomic augmentations which are predicted to manifest in more beneficial outcomes, based on existing linkages between equipment, equipment features, and ergonomics issues, as set out in the relational library 310.

In some embodiments, the predictive model 500 may be further configured to update the linkages in relational library 310. For example, certain equipment or equipment features may become recognized by predictive model 500 to be associated with certain ergonomic issues and benefits, which were not previously programmed into relational library 310. In such instances, the predictive model 500 may update, or cause recommendation engine 430 to update, the linkages in relational library 310. Updating a linkage may involve creating a linkage, removing a linkage, or modifying a weighting factor associated with a linkage.

In addition to ultimately making a recommendation for augmenting the workspace, the predictive model 500 may be referenced when monitoring workers in general, and to generate predictions of ergonomics risks. A particular set of user data 150 can thereby lead to a prediction that the patient will experience a workplace patient injury or discomfort in the future. The predictive model 500 may then be consulted before implementing preemptive intervention measures, such as replacing workplace equipment, or instituting a regimen of physical therapy, before an injury manifests.

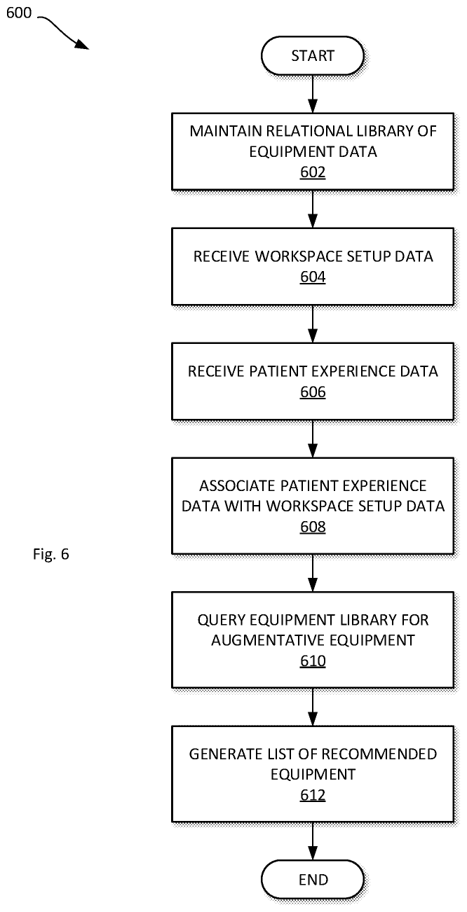

FIG. 6 is a flowchart showing a method 600 for augmenting a workspace, according to a non-limiting embodiment. Briefly, user data relating to a workspace and a patient's experience of the workspace is received, and with reference to a relational library of equipment data is used to identify deficiencies in the patient's workspace equipment. A recommendation for ameliorate the deficiencies is made by a recommendation engine. It is to be emphasized, however, that the blocks of method 600 need not be performed in the exact sequence as shown. Further, the method 600 is described as performed by a system and device discussed herein, but this is not limiting and the method can alternatively be performed by other systems and/or devices.

At block 602, a relational library of equipment data is maintained. As an example, relational library 310 is maintained on assessment server 300.

At block 604, patient workspace setup data is received. As an example, user data 150 includes data relating to the equipment 104 present in a workspace 102, and includes deficiencies identified in the equipment 104. The user data 150 is transmitted to assessment server 300 over network 110.

At block 606, patient experience data is received. As an example, user data 150 includes data relating to a patient's experience of using the equipment 104 present in a workspace 102, and includes discomforts the patient is experiencing, such as chronic pain in areas of the body. The user data 150 is transmitted to assessment server 300 over network 110.

At block 608, patient experience data is associated with workspace setup data. As an example, the relational library 310 may include linkages which indicate that an office chair without customizable lumbar support may be a contributory cause of chronic lower back pain. The recommendation engine 430 can take this linkage, in addition to other information, to associate the deficiency of lack of customizable lumbar support with the chronic lower back pain.

At block 610, an equipment library is queried for augmentative equipment. As an example, the relational library 310 may include entries for available office chairs which include customizable lumbar support, which the recommendation engine 430 may be programmed to recommend to ameliorate the patient's chronic lower back pain. Querying the relational library 310 may be iterated until a recommendation is made to address each deficiency.

At block 612, a list of recommended equipment is generated. As an example, the list of recommended equipment is generated by recommendation engine 430 from querying the relational library 310 for equipment satisfying each of the patient's identified deficiencies.

Although in the present embodiment, the augmentation to the workspace comprises a recommended list of equipment to ameliorate equipment deficiencies, it is also contemplated that in other embodiments, a relational database may be referenced to identify other interventional augmentations, such as physical therapy regimens, which are generally understood to treat certain patient discomforts, and recommendation of such additional augmentations may be made.

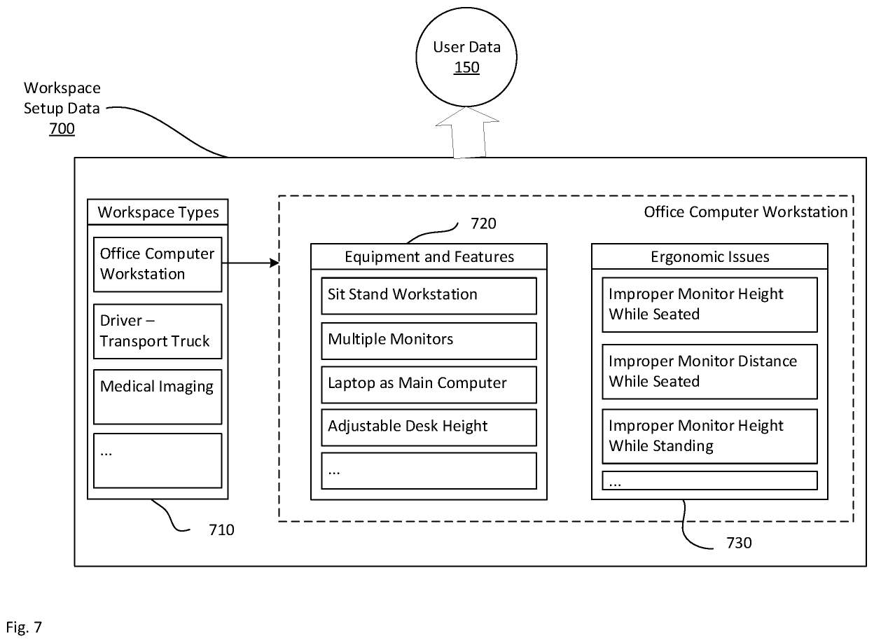

FIG. 7 is a schematic diagram showing a data structure layout of patient-provided workspace setup data 700, according to a non-limiting embodiment. Workspace setup data

Patient-provided workspace setup data 700 includes one or more workspace tables 710 having attributes indicating different types of workspaces, such as an office computer workstation, or a transport truck driver workstation. A workspace type can be selected by a patient during the course of an ergonomics assessment for inclusion of data related to equipment used in the workspace into user data 150. A one-to-many relationship may exist for each workspace type to features tables 720 having attributes indicating different features of the related workspace type. For example, an office computer workstation may be a sit-stand workstation, or have multiple monitors. The relevant workspace features are included in user data 150. A one-to-many relationship may exist for each workspace type to features ergonomics issues tables 730, having attributes indicating different ergonomics issues associated with the workspace type. For example, an office computer workstation may have improper monitor height while the user is seated. Again, a patient reports such setup information and provides records for tables 720 and 730 for inclusion into user data 150.

Records associated with each table 710, 720, 730, may be gathered during the course of an ergonomic assessment via a patient user interface manager 406 for inclusion in user data 150.

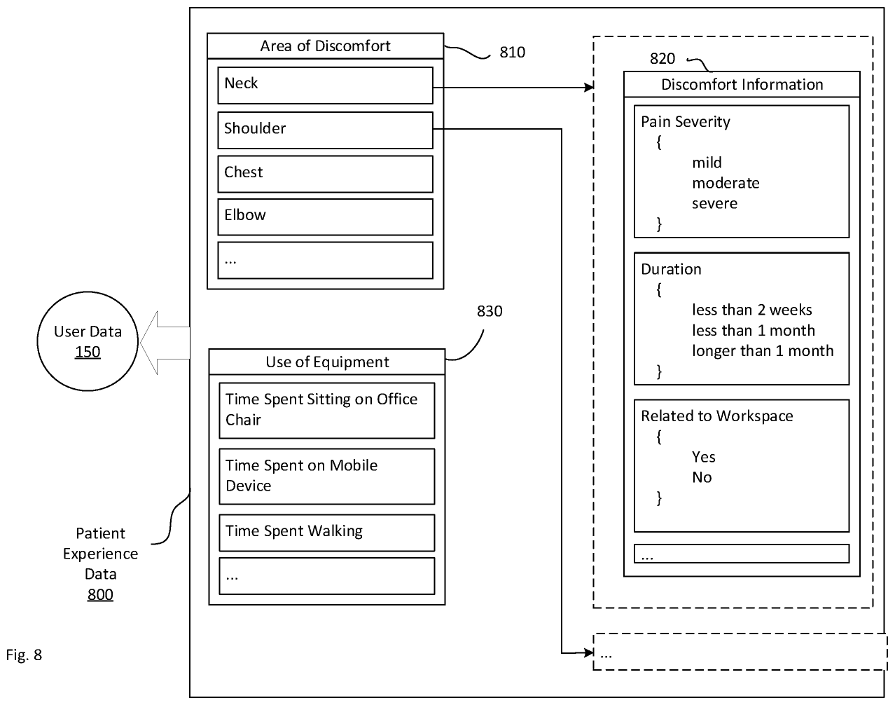

FIG. 8 is a schematic diagram showing a data structure layout of patient experience data 800, according to a non-limiting embodiment. Patient experience data 800 includes one or more discomforts tables 810 having attributes indicating different areas of discomfort experience by the patient, such in the neck or shoulder. An area of discomfort can be selected by a patient during the course of an ergonomics assessment for inclusion into user data 150. A one-to-many relationship may exist for each discomfort to discomfort information tables 820 having attributes indicating additional information related to the discomfort. For example, a discomfort in the neck may have mild to severe severity, and may be experienced for a certain frequency for a certain duration. Patient experience data 800 also includes one or more equipment use tables 830 having attributes indicating different manners in which a patient's time in a workspace may be spent. For example, a duration of time may be spent sitting in an office chair, and another duration of time may be spent using a mobile device. A patient providers records such experience information and provides records for tables 820 and 830 for inclusion into user data 150.

Records associated with each table 810, 820, 830, may be gathered during the course of an ergonomic assessment via a patient user interface manager 406 for inclusion in user data 150.

FIG. 9 is a schematic diagram showing a data structure layout of equipment feature data 900, according to a non-limiting embodiment. Equipment feature data 900 may be provided by experts knowledgeable about particular pieces of ergonomic equipment. Equipment feature data 900 includes one or more equipment tables 910 having attributes indicating different pieces of workspace equipment, such as a particular chair or a particular monitor platform used in an office. A one-to-many relationship may exist for each piece of equipment to features tables 920 having attributes indicating ergonomically-relevant features possessed by the associated piece of equipment. For example, a particular chair may include a sturdy five star base. A one-to-many relationship may exist for each piece of equipment to deficiencies tables 930 having attributes indicating ergonomically-relevant deficiencies possessed by or risks associated with the associated piece of equipment. For example, a particular chair may not have adjustable backrest support.

Records associated with each table 910, 920, 930, are maintained on assessment server 300 in relational library 310.

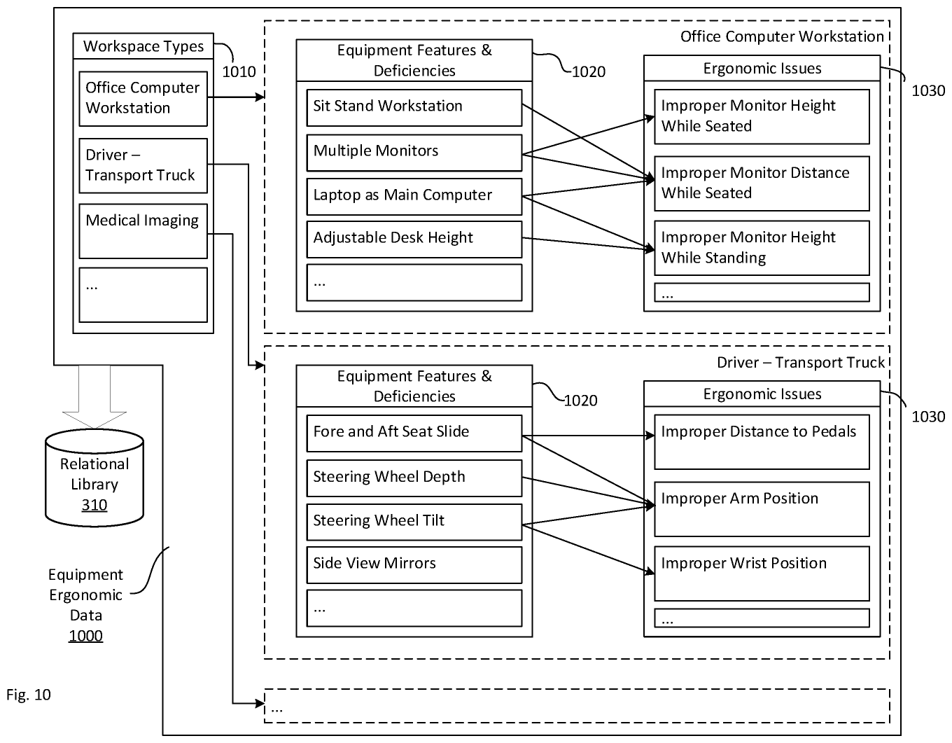

FIG. 10 is a schematic diagram showing a data structure layout of equipment ergonomic data 1000, according to a non-limiting embodiment. Equipment ergonomic data 1000 may be provided by experts knowledgeable about particular pieces of ergonomic equipment. Equipment ergonomic data 1000 includes one or more workspace types tables 1010 having attributes indicating different types of workspaces, such as an office computer workstation and a transport truck driver workstation. A one-to-many relationship may exist for each workspace type to features tables 1020 having attributes indicating different features of the related workspace type. For example, an office computer workstation may be a sit-stand workstation, or have multiple monitors. One-to-many, one-to-one, or many-to-one relationships may exist for each equipment feature or deficiency in table 1020 to a known ergonomics issue in ergonomics issues tables 1030. For example, it may be known to ergonomics experts, and the relationships between tables 1020 and 1030 may be designed, such that having multiple monitors may be an indicator of the ergonomic issue that improper monitor height while seated, or that improper monitor distance while seated, may be present.

Records associated with each table 1010, 1020, 1030, are maintained on assessment server 300 in relational library 310.

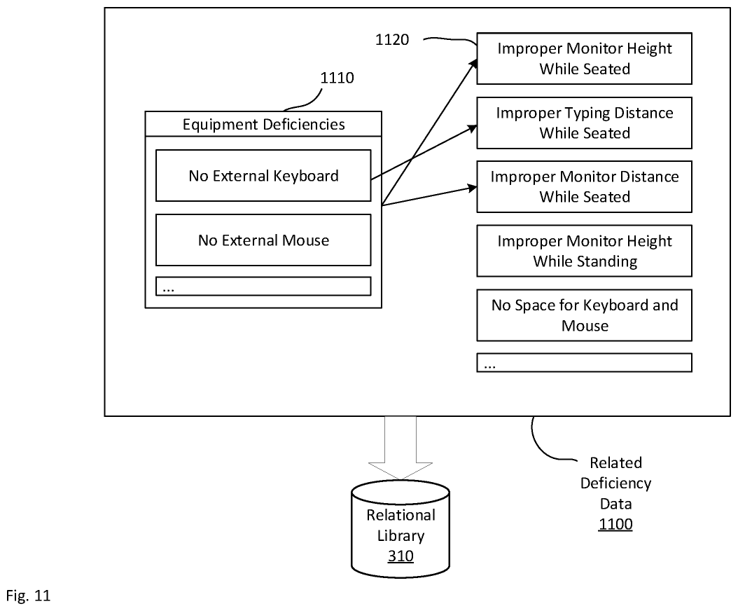

FIG. 11 is a schematic diagram showing a data structure layout of related deficiency data 1100, according to a non-limiting embodiment. Related deficiency data 1100 may be provided by experts knowledgeable about particular ergonomics concepts, including the relationships between various ergonomics issues. Related deficiency data 1100 includes one or more related deficiencies tables 1110 having attributes indicating different types of deficiencies 1120, such as lack of an external keyboard or lack of an external mouse. A one-to-many relationship may exist for each deficiency to other deficiencies. For example, having no external keyboard may be known to be associated with improper typing distance while seated. Having no external keyboard and no external mouse means that the patient is working directly from the laptop, which means the user cannot adjust monitor height or distance.

Records associated with each table 1110, 1130, and deficiencies 1120, are maintained on assessment server 300 in relational library 310.

Equipment feature data 900, equipment ergonomic data 1000, and related deficiency data 1100 may be linked in relational library 310, such that relationships between particular workspace types, equipment, features, and ergonomics issues, may be configured. Thus, when the recommendation engine 430 refers to relational library 310, it may then query for equipment to recommend which augments the deficiencies reported by the patient while avoiding the introduction of new deficiencies. Recommended equipment may vary based not only on workspace setup data, but on patient experience data, such that, for example, in some cases a particular piece of equipment may be suitable only where a patient reports a particular type of discomfort. Various algorithms may be used to match user data 150 with recommended augmentations

Although in the present embodiment, the recommendation augmentations to the workspace include additional workspace equipment, it is understood that the data structures above may be complemented by additional information related to further augmentative interventions, such as physical therapy. As such, a recommendation by recommendation engine 430 may include a recommended list of equipment and/or additional ergonomic interventions.

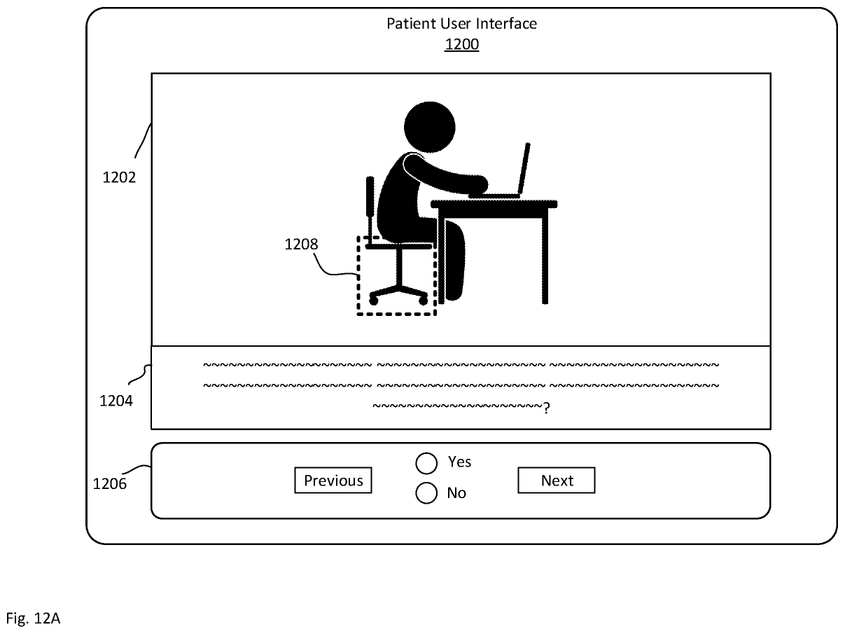

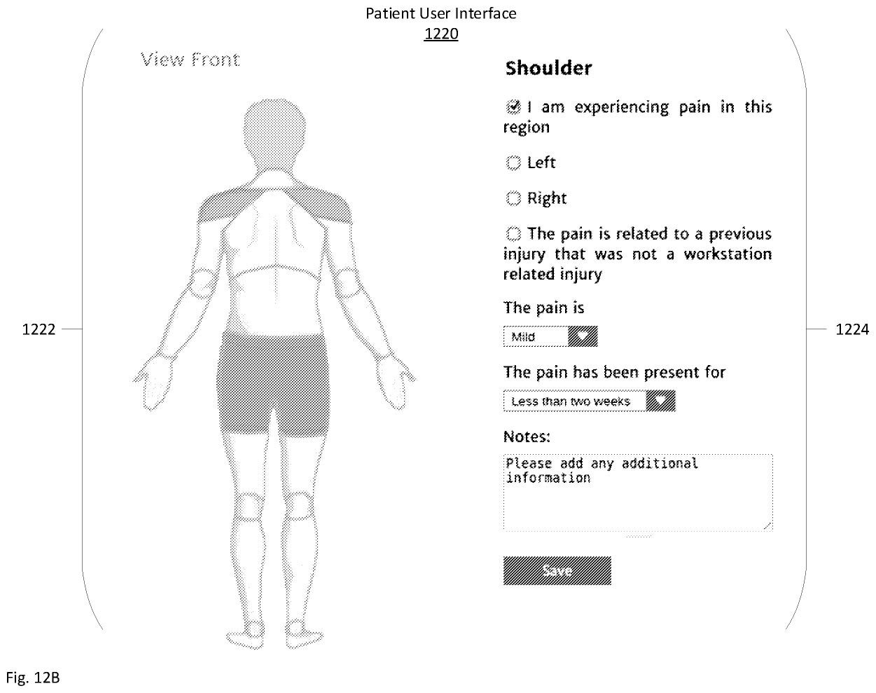

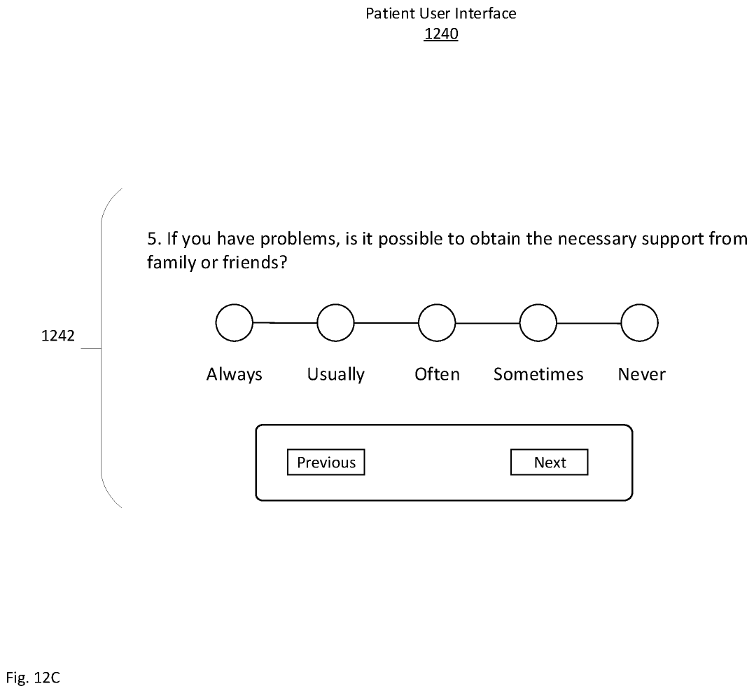

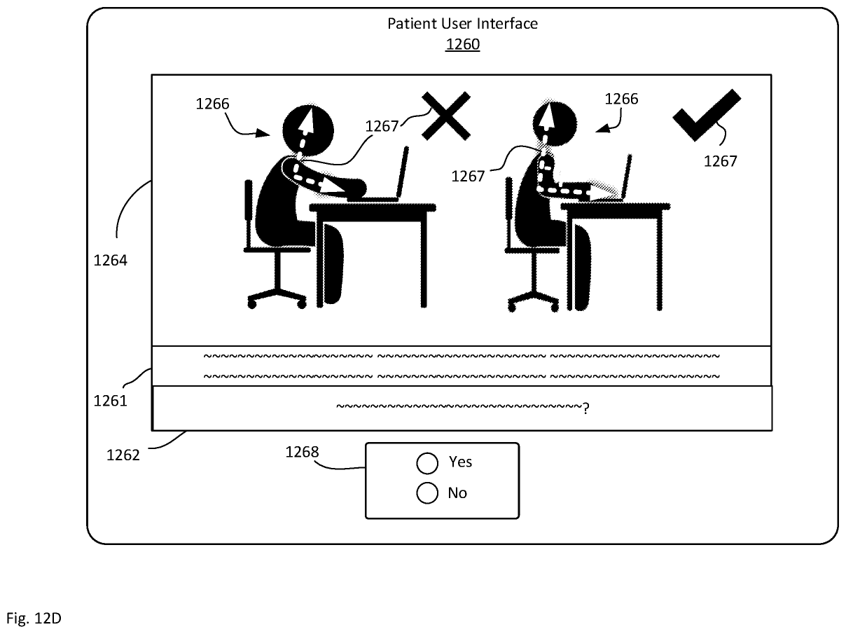

FIG. 12A is a schematic diagram of a patient user interface 1200, according to a non-limiting embodiment. Patient user interface 1200 includes an interrogative component 1204 for outputting a question from a question library 330, routed from assessment server 300. The questions are outputted in cooperation with question outputter 404, and are directed to obtaining patient workspace setup data related to a piece of equipment used in the workspace from the patient. An input component 1206 enables a user to input a response to the questions outputted, e.g. via input device 258. A graphical component 1202 displays a scene including a representation of the piece of equipment used in the workspace to which the question is related. The graphical component 1202 may include a highlighted portion 1208 to indicate a particular piece of equipment in the scene about which a question is being asked. The scene displayed changes with the question being asked, and is updated with information obtained through the ergonomics assessment. Thus, patient user interface 1200 provides for an intuitive method for a patient to provide ergonomically relevant information to the system. For example, where a user indicates that they have an office workspace with three external monitors, and the question outputted relates to whether the three monitors have monitor stands, the graphical component 1202 will display three monitors having monitor stands, and highlight the monitor stands. As another example, the question outputted may ask whether the user can adjust the monitors to proper viewing height, and the graphical component 1202 may demonstrate proper monitor height with three monitors. Where the patient responds that they cannot, then a potential workspace deficiency may then be identified and flagged.

FIG. 12B is a schematic diagram of a patient user interface 1220, according to a non-limiting embodiment. Patient user interface 1220 includes a graphical component 1222, and a mixed interrogative-input component 1224. In the present example, patient user interface 1250 is used to obtain patient experience data for inclusion into user data 150. In the present example, mixed interrogative-input component 1224 may ask questions and receive answers from a user regarding physical pains and/or discomforts which may be associated with the user's workspace. The graphical component 1222 may aid in directing the user's attention to a particular pain or discomfort being discussed.

FIG. 12C is a schematic diagram of a patient user interface 1240, according to a non-limiting embodiment. Patient user interface 1240 includes a mixed interrogative-input component 1242. In the present example, the patient user interface 1240 is used to obtain psychometric data to identify neurocognitive risk factors for inclusion into patient experience data and user data 150. In the present example, mixed interrogative-input component 1242 may ask questions and receive answers from a user regarding psychological well-being, including general contentment level, level of engagement with one's work, motivation, emotional well-being social well-being, and other related areas of questioning.

Studies have shown that psychometric data, including information about an individual's psychological well-being, is related to how an individual perceives musculoskeletal injuries, and the effect such injuries have on an individual. For example, studies have shown that although there may be no relationship between extent of computer use and chronic neck and shoulder pain, low social support in the workplace is associated with neck pain, and high-demand and low-control jobs are associated with shoulder pain. As another example, studies have shown that psychosocial factors may be modestly associated with new onset shoulder pain, and that monotony of work is a strong risk factor for new onset shoulder pain. As another example, studies have shown that psychological distress is an important predictor of onset forearm pain. Thus, a person in a poor neurocognitive state may be more susceptible to suffering a musculoskeletal injury, more likely to perceive such an impairment as more severe, and more likely to have such an impairment affect one's productivity in the workplace.

As such, the use of psychometric data as a predictor of ergonomic risk may be particularly useful in identifying candidates for ergonomic intervention, and thereby particularly useful in reducing lost productivity and worker absenteeism. This predictive ability can therefore be combined with the system described herein, whereby an individual at high ergonomic risk due to psychometric factors can be identified, and preventative measures can be taken, such as augmenting the workspace with new equipment, physiotherapy, or psychological therapy, to reduce ergonomic risks to the individual.

Psychological risk factors may be assessed using a questionnaire administered through patient user interface 1240. The same, or similar, questions may be repeated at intervals throughout an individual's use of a workspace, for example, once per year. The questionnaire can include questions related to the individual's job demands, such as workload, sensory demands, cognitive demands, as well as to the individual's job control, such as decision latitude and freedom in work, as well as to the individual's social support, including support from supervisors and support from colleagues. The questionnaire may also include questions related to the individual's personal characteristics, including physical characteristics such as age, gender, body mass, and height, as well as the individual's level of physical activity, personality type, and social network. Questions may also ask about the individual's level of job satisfaction, engagement with work, and stress levels. Responses to such questions may be obtained through mixed interrogative-input component 1242 for inclusion into user data 150, and included into predictive model 500.

In some embodiments, psychometric data may be obtained through means other than direct questioning. For example, patient user interface manager 406 may include programming instructions to record a patient's interaction with a patient user interface 1200, 1220, 1240, or 1260. Data relating to a patient's interaction, such as the speed at which the patient advances through questioning, the patient's movements of an input device such as a mouse, the overall length of time taken by the patient to complete the ergonomic assessment, may serve as predictors for a patient's psychological well-being, which may in turn serve as a predictor of the patient's susceptibility to ergonomic risk such as physical injury.

In some embodiments, psychometric data may be obtained through competitive test performance measurement. For example, to measure motivation, a patient may be asked to press a button as quickly as possible on a keyboard (e.g. input device 258) when a computer (e.g. patient device 200) instructs them to do so. The time delay between when a patient is instructed to press the button and when the button is actually pressed may serve as a useful predictor of the patient's level of engagement at work. A patient who presses the button within 1-2 seconds is likely to be highly motivated, whereas a demotivated person may be distracted, lethargic, or unmotivated, and may press the button within, for example, 3-5 seconds. Such competitive tests may be administered throughout the ergonomic assessment, and results may be included into user data 150, and included in predictive model 500.

FIG. 12D is a schematic diagram of a patient user interface 1260, according to a non-limiting embodiment. Patient user interface 1260 includes instructional component 1261 which conveys instructions or educational information about proper ergonomic use of equipment in the workspace to the user, and an interrogative component 1262 for outputting a question to the user regarding the instructions or educational information conveyed in instructional component 1261. For example, instructional component 1261 may include statements regarding proper sitting and arm positioning, and interrogative component 1262 may ask the user whether they are capable of assuming the instructed ergonomic position. In other embodiments, instructional component 1261 may include instructions or information regarding how to setup or adjust a particular piece of workspace equipment, and the interrogative component 1262 may ask a question regarding whether the user is able to comply with the instruction. Where the user is unable to comply, a deficiency in the workspace equipment may be identified and flagged.

The patient user interface 1260 also includes a graphical component 1264, which may display visual representations 1266 of the instruction or educational material being conveyed, or the question being asked. In some embodiments, the visual representations may include visual cues 1267 to guide the user through the instruction, education, or question, as the case may be. The patient user interface 1260 also includes an input component 1268 for receiving an answer to the question posed in interrogative component 1262.

Thus, an automated ergonomic assessment may include a patient using any combination of patient user interfaces 1200, 1220, 1240, and 1260. The system thereby provides a way to obtain ergonomically relevant information such as workspace setup data, patient experience data, and information about how a patient interacts with their workspace, in an intuitive way from the patient, while providing intuitive education and instruction as to proper ergonomic use of available workspace equipment.

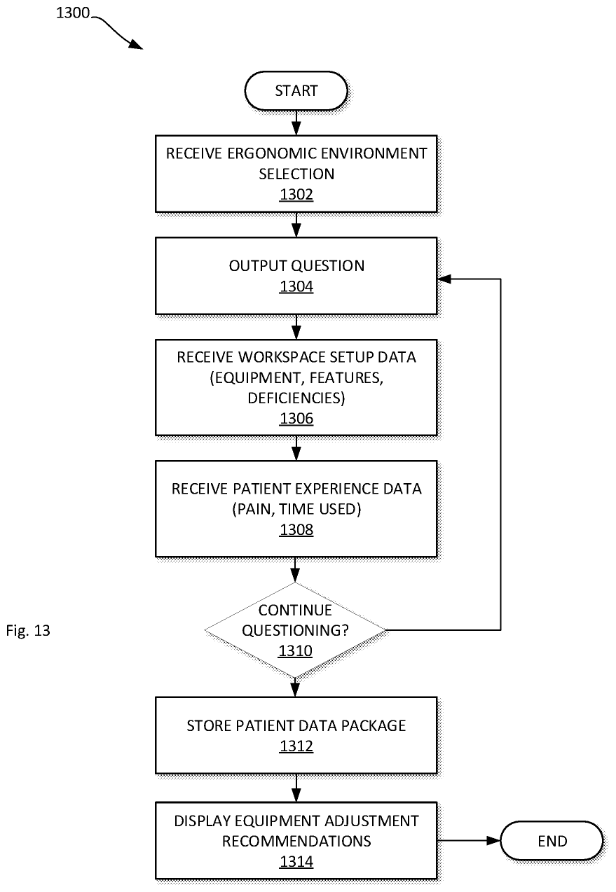

FIG. 13 is a flowchart showing a method 1300 performing an ergonomic assessment of a patient using a workspace, according to a non-limiting embodiment. Briefly, an ergonomic environment is selected, relevant questions are outputted to a user through a branching sequence of questions to obtain relevant information relating to the workspace setup and the patient's experience thereof. Immediate recommendations may be made as to how to improve the user's ergonomic situation with the equipment at hand. It is to be emphasized, however, that the blocks of method 1300 need not be performed in the exact sequence as shown. Further, the method 1300 is described as performed by a system and device discussed herein, but this is not limiting and the method can alternatively be performed by other systems and/or devices.

At block 1302, a selection of ergonomic environment is received. For example, a question may be outputted through patient user interface 1200 to ask the general category of workspace environments to which the ergonomic assessment will relate. A relevant subset of questions can then be selected for output.

At block 1304, a question is output to the user. For example, questions are output through interrogative component 1204 of patient user interface 1200.

At blocks 1306, 1308, in response to the question outputted at block 1304, a response is received. For example, a response is received through input component 1206 of patient user interface 1200.

At block 1310, it is determined whether to continue questioning. Generally, questioning may continue until the ergonomics assessment is complete, after all ergonomically relevant equipment has been identified in the workspace, and the features thereof have been identified. In some embodiments, the path of questioning may be determined in accordance with a branching sequence of questioning, as described in FIG. 14, below.

At block 1312, a patient data package is stored. For example, the patient data package comprises user data 150, and is transmitted for storage at assessment server 300. In some embodiments, user data 150 is stored at the patient device 200.

At block 1314, equipment adjustment recommendations are displayed. For example, equipment adjustment recommendations may be displayed through graphical component 1202 of patient user interface 1200. For example, patient user interface manager 406 may include logic to provide equipment adjustment recommendations directly to the user upon conclusion of an ergonomics assessment. For example, where the user has indicated a discomfort that is solvable with the equipment at hand, such as by adjusting a reclining angle of an office chair, the patient user interface manager 406 may directly output such a suggestion.

FIG. 14 is a branch diagram showing a branching sequence 1400 of questions for performing an ergonomic assessment, according to a non-limiting embodiment. The questions output through question outputter 404 may be filtered and selected in such a manner that previous answers to previous questions impact the next question in the sequence asked. In this way, irrelevant questions are not ouputted, and irrelevant information is not stored or transmitted.

For example, at 1402, a question asks whether the patient has a laptop computer as a main computer, and receives a positive response. Question outputter 404 may therefore skip questions related to a desktop computer acting as a main computer. Rather, question outputter 404 may select a branch of questioning relevant to the patient having a laptop as a main computer, such as about having external keyboards, mouse, and monitors.

For example, at 1410, a question asks whether the patient has an external monitor, and receives a negative response. Question outputter 404 may therefore skip questions related to adjusting an external monitor, and instead ask more contextually-relevant questions, such as questions related to the height of the laptop screen.

Referring again to FIGS. 12A, 12B, 13, and 14, it can be seen that a contextualized automated ergonomics assessment can be provided, which will now be described by way of example. First, the user may be presented with an instruction to assume proper arm position while seated, and the graphical component 1202 may indicate how to assume such a position. The user may then be asked via interrogative component 1204 whether they can assume the shown position. Where the user cannot, the user may respond so through input component 1206. At this point, the patient user interface manager 406 may identify whether the workspace presently contains equipment which may resolve this problem. For example, patient user interface manager 406 may recognize that the user has a chair with adjustable seat height which may be used to correct for arm position. Thus, the graphical component 1202 may output an instruction for the user to adjust their seat height to obtain proper arm position. Graphical component 1202 may include highlighted portions 1208 to direct the user's attention to the appropriate mechanisms of the chair for adjusting seat height. The user may then be asked again whether they can assume proper arm position while seated. If the user were to again reply in the negative, the patient user interface manager 406 may check whether the user has indicated the presence of an adjustable height desk. Where the user has not, the patient user interface manager 406 may again consider the next possible equipment which may solve the setup issue, such as an adjustable keyboard tray height feature. If, for example, the user has the adjustable keyboard tray height feature, but where the user also has an issue flagged that they have no external mouse, the patient user interface manager 406 may recognize that the user cannot be working from a keyboard tray. Where there are no other possible pieces of equipment that may resolve the setup issue given the current workspace equipment, the patient user interface manager 406 may flag the setup issue for inclusion into user data 150. The recommendation engine 430 may be able to recommend available equipment which is able to remedy the setup issue.

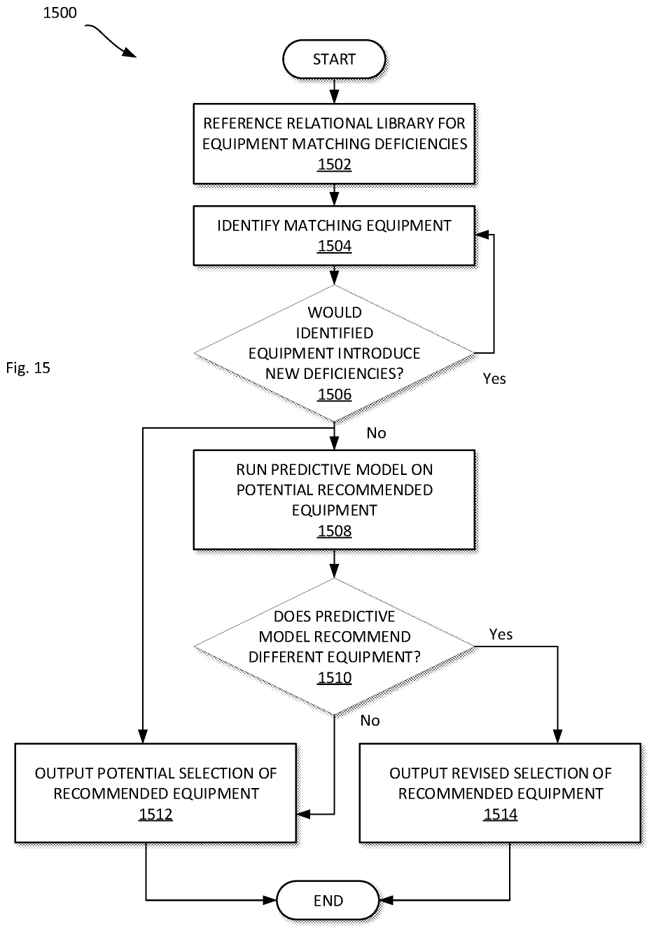

FIG. 15 is a flowchart showing a method 1500 of generating recommended equipment for a workspace, according to a non-limiting embodiment. Briefly, a relational library is referenced for equipment satisfying a particular set of deficiencies of a patient, and a predictive model is executed to predict improvement in the patient's circumstances if a particular recommendation is made. The best set of equipment for the patient is ultimately determined and recommended. It is to be emphasized, however, that the blocks of method 1500 need not be performed in the exact sequence as shown. Further, the method 1500 is described as performed by a system and device discussed herein, but this is not limiting and the method can alternatively be performed by other systems and/or devices.

At block 1502, a relational library is referenced for equipment matching a set of deficiencies. For example, user data 150 received at assessment server 300 includes several identified deficiencies, such as lack of a chair with proper armrest height, and a lack of a footrest. The recommendation engine 430 references relational library 310 for available equipment which satisfy these deficiencies.

At block 1504, equipment satisfying the deficiencies for which the reference was made at block 1502 are identified.

At block 1506, it is determined whether the matched equipment identified at block 1504 would introduce new deficiencies to the workspace if the equipment would be used. For example, the use of a particular new chair with proper armrest height may lack proper lumbar support, which the user data 150 has also indicated is an issue. The recommendation engine 430 can then iterate through the relational library 310 to identify matching equipment which would satisfy deficiencies without introducing new ones.

Following block 1506, a list of recommended equipment may then be outputted. For example, following completion of an ergonomics assessment, and following reference of the relational library 310 by the recommendation engine 430, a patient user interface 1200 may output a list of recommended equipment which the patient may be able to obtain. Alternatively, or together, the recommended equipment may be outputted to a manager device 120 to determine whether the recommended equipment is obtained.

Also following block 1506, a predictive model may be executed to determine the expected outcomes of recommended augmentations to a workspace. For example, the predictive model 500, trained with training library 320, may recognize that a different selection of recommended equipment has been shown to improve ergonomic outcomes for patients in circumstances similar to the circumstances of the patient undergoing the present ergonomics assessment. The predictive model 500 may determine, as in block 1510, that a different set of equipment may be better recommended, based on data related to previous recommended augmentations and differential outcomes thereof, stored in training library 320. Where the original selection of potentially recommended equipment is unchanged, the list may be outputted at block 1512. Where a different set of equipment may be better recommended, the revised selection of recommended equipment is outputted at block 1514.

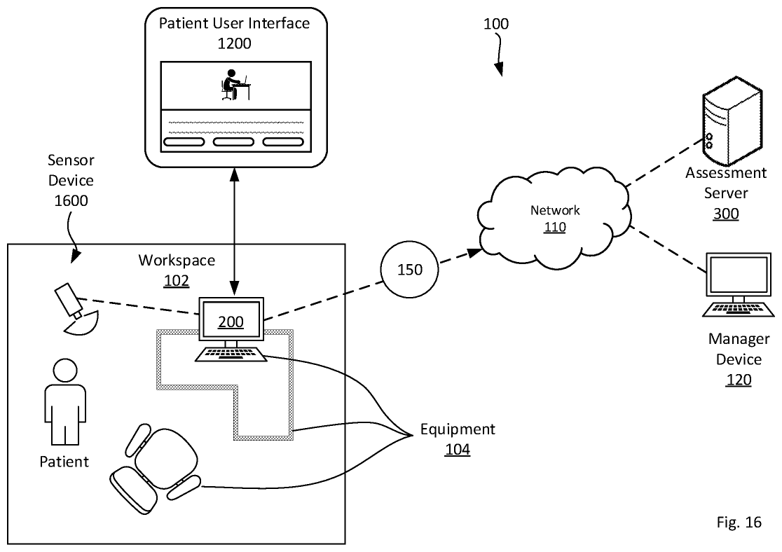

FIG. 16 is a schematic diagram of another embodiment of system 100, wherein the system 100 further includes one or more sensor devices 1600, according to a non-limiting embodiment. For description of the system 100, workspace 102, equipment 104, patient device 200, user data 150, network 110, assessment server 300, patient user interface 1200, and manager device 120, reference may be had to the description in FIG. 1. The system 100 includes a workspace, indicated generally at 102.

In the present embodiment, the sensor device 1600 is configured and oriented to capture sensor data from the workspace 102, to be relayed to patient device 200 for inclusion into user data 150. The sensor device 1600 may include a camera configured to recognize specific pieces of equipment 104 present in workspace 102. The sensor device 1600 may be programmed to relay imagery to a computing system, such as assessment server 300, patient device 200, or another computing system, where image recognition may be conducted on the imagery captured by sensor device 1600 to recognize equipment 104.

In some embodiments, the sensor device 1600 may include other sensing equipment, such as Radio Frequency Identification (RFID) or Near-field Communication (NFC) readers for detecting RFID or NFC tags on equipment 104 present in the workspace 102.

The sensor device 1600 may thereby supplement or replace user input through patient device 200. Further, specific features of equipment 104 may be detected as being present or improperly configured for proper ergonomic use, or damaged. The data captured by sensor device 1600 may be incorporated into training library 320, recommendation engine 430, and/or predictive model 500.

Returning again to FIG. 5, a discussion of the statistical learning methods which may be executed in accordance with the predictive model 500 are discussed. Generally, several statistically learning methods may be applied via predictive model 500, and the examples provided herein are exemplary only. As one example, predictive model 500 may be used to predict the number of discomforts a user will have based on ergonomic issues identified and the time spent using workspace equipment. In this example, where an output variable is being sought, a supervised learning method is appropriate. In this example, the input variables are categorical, either present or not present, and the time spent inputs are continuous. In this example, a statistical learning method appropriate to make this prediction is a support vector machine. As such, the predictive model 500 may comprise a machine learning model, and more particularly, a support vector machine.

Application of a support vector machine to the present system involves feature selection. In the present example, since the system is aware of ergonomic issues the user faces in the workspace, then the time categories, e.g. “Sitting while at home, commuting and at the office” and “Computer work out of office” may not be useful in increasing the accuracy of the prediction. As such, these time categories may be removed from consideration. In the present example, where certain ergonomic issues are known to have little impact on a user's discomfort, such as “Unstable chair” and “Glare on Monitor”, are identified, such issues may also be removed from consideration for the purpose of predictive analysis. In the present example, the user's time may be split between sitting and standing, and a user with a sit-stand workstation they may experience sit-stand related ergonomic issues. As such, the presence of either a sitting or standing setup issue may not reflect the same amount of time exposed to that issue as someone working in a sitting workstation who has a seated related issue. This incongruence may be address via feature engineering.

Feature engineering is the process by which inputs are transformed through, scaling, decomposition or aggregation, to provide more meaningful inputs to the system and often prevents the problem space from becoming too sparse. In the present example, the ergonomic issues related to standing and sitting are scaled to reflect the proportion of time they spend sitting versus standing, thus avoiding results of any predictions being skewed for users using sit-stand workstations. Thus, ergonomic setup issues may in fact be best treated on a continuum, e.g. as a value between 0 and 1 representing the proportion of time spent sitting versus standing, rather than categorical. Further, the actual time spent may be scaled to be represented by the fraction of a twenty-four hour day they represent, thus avoiding the time categories having undue leverage in an analysis. In the present example, where there may be significantly different levels of correlation between setup issues and discomforts, one may perform a linear regression analysis on each ergonomic issue with relation to the number of discomforts a user experiences, and exclude the inputs which do not exhibit an appropriate value of statistical significance. This operation avoids making the decision space unnecessarily sparse. For example “Insufficient leg space” may not influence the number of discomforts a user has, and is excluded if it is found to in fact be statistically insignificant.

The predictive model 500 may then be tuned with the above feature selection and feature engineering, and trained on training library 320. The predictive model 500, trained in the manner above, may yield a prediction of X discomforts based on provided user data 150. Thus, the predictive model 500 may be used to identify actionable insights. Several other analytical methods may be carried out by predictive model 500.

Thus, it can be seen that the present disclosure provides a method and system enabling a patient to receive a contextual and interactive automated ergonomic assessment. The ergonomic assessment may quickly provide the patient with immediate ergonomic assistance, education and instructing the patient to make equipment adjustments to the patient's existing workspace equipment via a user interface showing representations of the patient's workspace including visual cues to guide the patient. The ergonomic assessment may also result in augmentations to the workspace, including additional workspace equipment including features which address deficiencies identified in the patient's existing workspace equipment. The system includes a relational library for identifying such suitable equipment, and includes a recommendation engine which may execute a predictive model applying machine learning to gather actionable insights or make better-informed recommendations for improving a patient's workspace. Predictions may be made regarding the patient's susceptibility to ergonomic risk based on psychometric data. The system can integrate patient devices with manager devices to provide for large-scale management of ergonomics in an organization. The use of such systems can thus lead to cost savings for organizations using the system due to reduce patient injury, reduced absenteeism, and reduced insurance costs.

While the implementations discussed herein are directed to specific implementations of the system, it will be understood that combinations, sub-sets, and variations of the implementations are within the scope of the present disclosure.

The scope of the claims should not be limited by the embodiments set forth in the above examples, but should be given the broadest interpretation consistent with the description as a whole.

1. A system for ergonomically augmenting a workspace, the system comprising:
a patient user interface for receiving workspace setup data and patient experience data, the workspace setup data including equipment data related to equipment used in a workspace, the patient experience data relating to patient use of said equipment;
a memory, the memory storing programming instructions and a library of equipment data, the library of equipment data relating equipment identity and equipment features; and
a processor in communication with the memory and configured to execute the programming instructions to execute a recommendation engine, wherein the recommendation engine is configured to:
associate the patient experience data with the workspace setup data in the library of equipment data to identify deficiencies of the equipment used in the workspace; and
generate a recommended augmentation which ameliorates the deficiencies of the equipment used in the workspace.

2. The system of claim 1, wherein the memory stores a training library comprising workspace setup data and patient experience data, the training library relating workspace setup data to patient experience data and to outcomes of recommended augmentations, and wherein the recommendation engine generates a recommended augmentation according to a predictive model based at least in part on the training library.

3. The system of claim 2, wherein the predictive model comprises a machine learning model trained with the training library to recognize differential outcomes in patient experience data based at least on the outcomes of recommended augmentations.

4. The system of claim 3, wherein the machine learning model comprises a support vector machine.

5. The system of claim 2, wherein the predictive model comprises a machine learning model trained with the training library to predict ergonomic risk.

6. The system of claim 5, wherein the patient experience data comprises psychometric data, and wherein the predictive model is trained with the psychometric data to predict susceptibility to ergonomic risk.

7. The system of claim 6, wherein at least a portion of the psychometric data is obtained by recording interaction with the patient user interface.

8. The system of claim 6, wherein the recommended augmentation includes psychological therapy for a patient using the workspace.

9. The system of claim 1, wherein the recommended augmentation includes recommended equipment for the workspace having equipment features which ameliorate the deficiencies of the equipment used in the workspace.

10. The system of claim 9, wherein the recommendation engine is configured to exclude from recommendation the augmentation of recommended equipment which would introduce new deficiencies to the equipment used in the workspace.

11. The system of claim, where in the processor is further configured to receive workspace setup data via a sensor device configured to identify equipment present in the workspace.

12. The system of claim 1, wherein the recommended augmentation includes a recommended regimen of physical therapy for a patient using the workspace.

13. The system of claim 1, wherein the memory stores a question library, and wherein the patient user interface comprises:
an interrogative component for outputting a question from the question library, the question directed to obtaining patient workspace setup data or patient experience data related to a piece of equipment used in the workspace from a patient;
a graphical component for displaying a representation of the piece of equipment used in the workspace to which the question is related, and for displaying visual cues to guide the patient to answer the question; and
an input component for receiving a response to the question.

14. The system of claim 13, wherein the graphical component displays a representation of the workspace, the representation of the workspace generated from the workspace setup data, the graphical component further displaying a highlighted portion indicating the representation of the piece of equipment used in the workspace to which the question is related.

15. The system of claim 13, wherein the interrogative component is configured to output questions according to a branching sequence of questioning whereby a particular branch of questions is output depending on a previous answer to a previous question of a previous branch of questions.

16. The system of claim 1, wherein the system further comprises a manager user interface, and wherein the processor is further configured to display the workspace setup data and patient experience data and the recommended augmentation to the manager user interface.

17. The system of claim 1, wherein the system further comprises a computer network, a network interface in communication with the processor, a patient device running the patient user interface and in communication with the network interface via the computer network, and a manager device running the manager user interface and in communication with the network interface via the computer network.

18. A method for augmenting a workspace, the method comprising:
maintaining a library of equipment data, the library of equipment data relating equipment identity and equipment features;
receiving workspace setup data, the workspace setup data including equipment data related to equipment used in a workspace;
receiving patient experience data, the patient experience data relating to patient use of said equipment;
associating the patient experience data with the workspace setup data in the library of equipment data to identify deficiencies of the equipment used in the workspace; and
generating a recommended augmentation which ameliorates the deficiencies of the equipment used in the workspace.

19. The method of claim 18, wherein the generating the recommended augmentation comprises executing a predictive model trained with a training library relating workspace setup data to patient experience data and to outcomes of recommended augmentations.

20. The method of claim 19, wherein the predictive model comprises a machine learning model trained with the training library to recognize differential outcomes in patient experience data based on at least the outcomes of recommended augmentations.

21. (canceled)

22. (canceled)

23. (canceled)

24. (canceled)

25. (canceled)

26. (canceled)

27. (canceled)

28. (canceled)

29. (canceled)
